(19) United States
(12) Patent Application Publication
(10) Pub. No.: US 2026/0115013 A1
(43) Pub. Date: Apr. 30, 2026

(54) INFLATABLE MEDICAL DEVICE WITH ONE OR MORE TROUBLESHOOTING FEATURES

(71) Applicant: Boston Scientific Scimed, Inc., Maple Grove, MN (US)

(72) Inventors: Brian P. Watschke, Minneapolis, MN (US); Natalie Ann Borgos, Roseville, MN (US); Sarah Prozeller, Boxboro, MA (US); Matthew Johnson, Lino Lakes, MN (US); James F. Hiebert, Delano, MN (US)

(21) Appl. No.: 19/364,388

(22) Filed: Oct. 21, 2025

Related U.S. Application Data
(60) Provisional application No. 63/712,750, filed on Oct. 28, 2024.

(51) Int. Cl. A61F 2/48; A61F 2/00; A61F 2/26

(52) U.S. Cl. CPC A61F 2/482; A61F 2/0027; A61F 2/26; A61F 2250/0002; A61F 2250/0004

(57) ABSTRACT

An inflatable medical device may include an inflatable member. An inflatable medical device may include a fluid reservoir. An inflatable medical device may include an electronic pump device configured to be fluidly connected to the inflatable member and the fluid reservoir. The electronic pump device configured to execute operations, including inflating the inflatable member by transferring fluid from the fluid reservoir to the inflatable member, detecting that the inflatable member has been inflated for a period of time greater than a threshold level, and transmitting, via an antenna of the electronic pump device, a deflate signal to an external device. The deflate signal, when received at the external device, is configured to cause a deflate notification configured to be displayed on a display of the external device.

### CROSS-REFERENCE TO RELATED APPLICATION

This application claims priority to U.S. Provisional Patent Application No. 63/712,750, filed on Oct. 28, 2024, entitled “AN INFLATABLE MEDICAL DEVICE WITH ONE OR MORE TROUBLESHOOTING FEATURES”, the disclosure of which is incorporated by reference herein in its entirety.

### TECHNICAL FIELD

This disclosure relates generally to an inflatable medical device with one or more troubleshooting features.

### BACKGROUND

Some inflatable medical devices include a manual pump, physically operated by a user, to transfer fluid between an inflatable member and a fluid reservoir.

### SUMMARY

In some aspects, the techniques described herein relate to an inflatable medical device including: an inflatable member; a fluid reservoir; and an electronic pump device configured to be fluidly connected to the inflatable member and the fluid reservoir, the electronic pump device configured to execute operations, the operations including: inflating the inflatable member by transferring fluid from the fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

In some aspects, the techniques described herein relate to an inflatable medical device including: an inflatable member; a fluid reservoir; and an electronic pump device configured to be fluidly connected to the inflatable member and the fluid reservoir, the electronic pump device configured to execute operations, the operations including: inflating the inflatable member by transferring fluid from the fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

In some aspects, the techniques described herein relate to a method including: inflating, by an electronic pump device, an inflatable member by transferring fluid from a fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

In some aspects, the techniques described herein relate to a non-transitory computer-readable medium storing executable instructions that cause at least one processor to execute operations, the operations including: inflating, by an electronic pump device, an inflatable member by transferring fluid from a fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

### BRIEF DESCRIPTION OF THE DRAWINGS

FIG. 1A illustrates an example of a system for implementing one or more troubleshooting features of an inflatable medical device according to an aspect.

FIG. 1B illustrates an example of an external device for controlling an inflatable medical device according to an aspect.

FIG. 1C illustrates an inflatable medical device with an inflatable member, a fluid reservoir, and an electronic pump device according to an aspect.

FIG. 2 illustrates an exploded view of an electronic pump device according to an aspect.

FIG. 3 illustrates a perspective of an inflatable penile prosthesis according to an aspect.

FIG. 4 illustrates an example of an artificial urinary sphincter device according to an aspect.

FIG. 5 illustrates a flowchart depicting example operations of implementing one or more troubleshooting features of an inflatable medical device according to an aspect.

### DETAILED DESCRIPTION

This disclosure relates to a system with an inflatable medical device having an electronic pump device for automatically transferring fluid between a fluid reservoir and an inflatable member. In some examples, the inflatable member includes an inflatable cuff configured to be implanted around the urethra. In some examples, the inflatable medical device is a penile prosthetic, and the inflatable member is a pair of cylinders. The system may implement one or more troubleshooting features during use of the inflatable medical device and/or during implantation of the inflatable medical device.

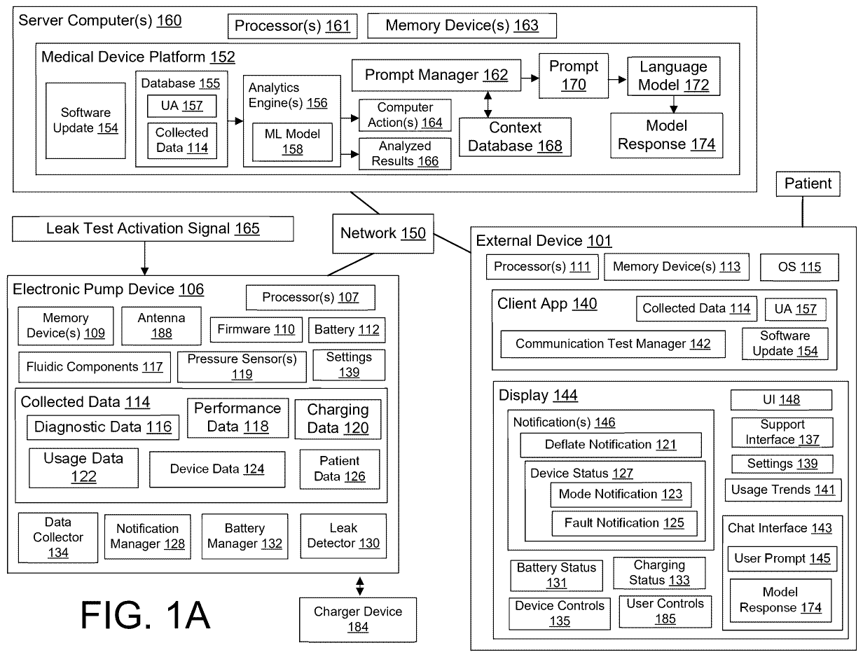

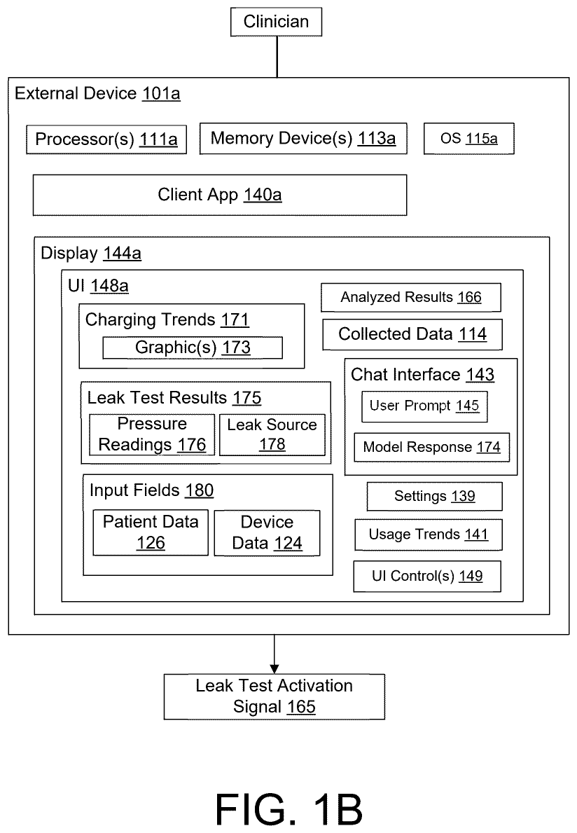

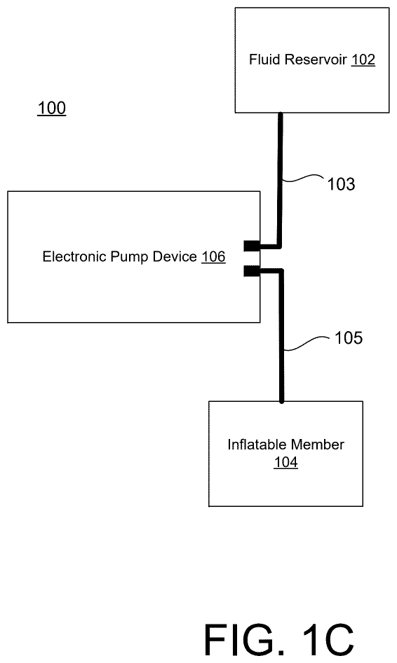

FIGS. 1A to 1C illustrates an inflatable medical device 100 according to an aspect. In some examples, the inflatable medical device 100 is an artificial urinary sphincter device. In some examples, the inflatable medical device 100 is an inflatable penile prosthesis. However, the inflatable medical device 100 may include any type of medical device that transfers fluid between components of the inflatable medical device 100. The inflatable medical device 100 may implement one or more troubleshooting features during use of the inflatable medical device and/or during implantation of the inflatable medical device

The inflatable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressurize the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable device 100 includes a single pressurized balloon. In some examples, the inflatable medical device 100 includes two or more pressurized balloons. The pressure in the inflatable member 104 may be generated by the fluid reservoir 102.

The implantable device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 includes one or more processors 107 and one or more memory devices 109. The memory device(s) 109 may store executable instructions that when executed by the processor(s) 107 to execute operations discussed herein with respect to the electronic pump device 106. The memory device(s) 109 may store firmware 110. In some examples, the firmware 110 may be an operating system of the electronic pump device 106. In some examples, the electronic pump device 106 includes a data collector 134 configured to receive and/or generate information (e.g., collected data 114) about the inflatable medical device 100 (and, in some examples, about a user of the inflatable medical device) and store the information (or a portion thereof) in the memory device(s) 109. The memory device(s) 109 may store collected data 114, which may be performance data 118, diagnostic data 116, charging data 122, usage data 122, device data 124, and/or patient data 126.

The electronic pump device 106 includes fluidic components 117 configured to enable the transfer of fluid between the inflatable member 104 and the fluid reservoir 102. The fluidic components 117 may include one or more pumps (e.g., electronic pumps, e.g., pumps operated by electrical signals) and one or more fluid valves. The electronic pump device 106 includes one or more pressure sensors 119 configured to generate pressure readings about a pressure in the inflatable member 104 and/or a pressure in the fluid reservoir. In some examples, the pressure sensor 119 includes a first pressure sensor connected to the inflatable member 104, which generates pressure readings about the pressure in the inflatable member 104 according to a sampling rate. In some examples, the pressure sensor 119 includes a second pressure sensor connected to the fluid reservoir 102, which generates pressure readings about the pressure in the fluid reservoir 102 according to a sampling rate.

The electronic pump device 106 may include an antenna 188 configured to wirelessly transmit (and receive) wireless signals from an external device 101. In some examples, the electronic pump device 106 communicates with the external device 101 over a network 150, which may be a Wi-Fi connection, mobile network connection, or a short-range communication network such as Bluetooth. In some examples, the electronic pump device 106 can communicate directly with a medical device platform 152 executable by one or more server computers 160. The server computer(s) 160 includes one or more processors 161 and one or more memory devices 163. The memory device(s) 163 may store executable instructions that cause the processor(s) 161 to execute operations with respect to the medical device platform 152 discussed herein. The memory device(s) 163 may store one or more databases 155. The databases 155 may store user accounts 157 for users of the medical device platform 152. The databases 155 may store collected data 114 (or a portion thereof). In some examples, the memory device(s) 163 may store a context database 168, which may be used to augment user prompts 145 for a language model 172. In some examples, the electronic pump device 106 and the external device 101 communicate with each other via a short-range communication network, and the external device 101 and the medical device platform 152 communicate with each other via a Wi-Fi or mobile network communication network.

The external device 101 may be any type of component that can communicate with the electronic pump device 106. The external device 101 may be a computer, smartphone, tablet, pendant, wearable device (e.g., smartwatch, wristband), key fob, etc. A user may use the external device 101 to control the inflatable medical device 100. In some examples, the user may use the external device 101 to inflate or deflate the inflatable member 104. As shown in FIG. 1A, the external device 101 is a user device of a patient of the inflatable medical device 100. In some examples, the external device 101 is a computing device such as a mobile device (e.g., a smartphone). The external device 101 includes one or more processor(s) 111 and one or more memory device(s) 113. The memory device(s) 113 may store executable instructions that cause the external device 101 (e.g., a client application 140) to execute operations discussed herein with respect to the external device 101.

In some examples, the external device 101 includes a client application 140 configured to communicate with the electronic pump device 106 and the medical device platform 152. In some examples, the client application 140 is installed on an operating system 115 of the external device 101. In some examples, the client application 140 is the operating system 115 or a sub-component of the operating system 115. In some examples, the client application 140 is a mobile (native) application. In some examples, the client application 140 is a web application executable by a browser application. In some examples, the client application 140 is a website of the medical device platform 152. The client application 140 is configured to display a user interface 148 on a display 144 of the external device 101. In some examples, the client application 140 is associated with a user account 157, which stores information about the patient and/or the inflatable medical device 100, which may include settings 139 of the inflatable medical device 100. In some examples, the medical device platform 152 stores the user accounts 157 in a database 155. In some examples, the user account 157 may store charging data 120, usage data 122, device data 124, and/or patient data 126, which may be generated by the electronic pump device 106.

The user interface 148 may include device controls 135 for controlling the inflation and deflation of the inflatable member 104. In some examples, the device controls 135 are UI elements. In response to selection of a first device control, the client application 140 may transmit an inflation signal to the electronic pump device 106, which causes the electronic pump device 106 to inflate the inflatable member 104 (e.g., by activating one or more pumps and/or one or more valves) by transferring fluid from the fluid reservoir 102 to the inflatable member 104. In response to selection of a second device control, the client application 140 may transmit a deflation signal to the electronic pump device 106, which causes the electronic pump device 106 to deflate the inflatable member 104 (e.g., by activating one or more pumps and/or one or more valves) by transferring fluid from the inflatable member 104 to the fluid reservoir 102.

The client application 140 may enable the user to select or adjust one or more settings 139 associated with the inflatable medical device 100. The settings 139 may include target inflation pressure, target deflation pressure, pump rate, and/or other settings that can control the operation of the inflatable medical device 100. In some examples, the settings 139 include an inflation delay (e.g., a time delay between selecting to change the inflation pressure and the action of transferring fluid). The client application 140 may display one or more settings 139 on the user interface 148. In some examples, when a setting 139 is adjusted, the client application 140 may communicate with the electronic pump device 106 to update the setting 139 at the electronic pump device 106 and/or the medical device platform 152.

In some examples, the external device 101 is an external device 101a associated with a clinician, as shown in FIG. 1B. The external device 101a may be an example of the external device 101 and may include any of the details discussed with reference to the external device 101. The external device 101a includes one or more processors 111a, one or more memory devices 113a, and an operating system 115a. The external device 101a includes a client application 140a executable (or defined) by the operating system 115a. The client application 140a may display a user interface 148a on a display 144a of the external device 101a. The client application 140a may be an example of the client application 140 and may include any of the details discussed with reference to the client application 140. In some examples, the client application 140 is a patient application, and the client application 140a is a clinician application. In some examples, the client application 140a includes different or additional features as compared to the patient application. For example, a clinician may use the client application 140a to create a user account 157 for a patient, enter patient data 126 and/or device data 124, determine or adjust one or more settings 139, and/or view certain information, which may not be available with the patient application.

In some examples, a clinician can enter or scan patient data 126 and device data 124 using the client application 140a. For example, the client application 140a may display a user interface 148a with one or more input fields 180 to enter patient data 126 and device data 124. In some examples, the clinician can use the client application 140a to scan a barcode of the inflatable medical device 100, which can automatically populate the user interface 148 with the device data 124. For example, the client application 140a may enable the external device's camera to scan a barcode, which may identify the inflatable medical device 100 from the context database 168 and/or the database 155. In some examples, the client application 140a may provide the physician (or other health care provider or sales representative present during the surgery and/or providing support) the ability to enter pertinent information regarding the patient and/or implanted product. In some examples, the client application 140a may transmit the patient data 126 and the device data 124 to the electronic pump device 106 for storage in the memory device 109. In some examples, the clinician may use the client application 140a to initiate the creation of a user account 157 at the medical device platform 152, and the patient data 126 and the device data 124 may be stored in the user account 157 at the database 155. In some examples, the patient data 126 and the device data 124 are stored at the memory device 109 of the electronic pump device 106. The patient data 126 may include personal information such as name, birth date, age, and/or other information about the patient. The device data 124 may identify the type of inflatable medical device 100, including cylinder size and type.

In some examples, the electronic pump device 106 includes a notification manager 128 configured to cause the external device 101 (e.g., the client application 140) to display one or more notifications 146 about issues (e.g., troubleshooting issues) relating to use of the electronic pump device 106. For example, the user may use a device control 135 to cause the electronic pump device 106 to inflate the inflatable member 104 (e.g., activating one or more pumps or values to transfer fluid from the fluid reservoir 102 to the inflatable member 104).

The notification manager 128 may detect that the inflatable member 104 has been inflated for a period of time greater than a threshold level. For example, the notification manager 128 uses a timer to determine the amount of time that the inflatable member 104 has been inflated, e.g., after the inflatable member 104 has reached a target pressure level. If the amount of time is equal to or greater than a threshold level, the notification manager 128 may transmit, via the antenna 188 of the electronic pump device 106, a deflate notification signal to the external device 101. The deflate notification signal, when received at the external device 101, is configured to cause a deflate notification 121 to be displayed on a display 144 of the external device 101. For example, the deflate notification signal causes the client application 140 to generate a deflate notification 121, which is displayed on the device's display 144. In some examples, the deflate notification 121 is a push notification. The deflate notification 121 may include information that the inflatable member 104 should be deflated. In some examples, the deflate notification 121 includes one or more controls, which, when selected, causes the client application 140 to launch and display an interface with the device controls 135 (e.g., to deflate the inflatable member 104) or causes the client application 140 to generate and transmit a deflate signal to the electronic pump device 106 to initiate deflation of the inflatable member 104.

In some examples, in response to a user selection of the deflate notification 121 (or a user selection of a control on the deflate notification 121), the external device 101 causes the client application 140 to launch and render the user interface 148 with the device controls 135. Then, the user may select a deflate control (e.g., one of the device controls 135) on the user interface 148, which causes the client application 140 to generate and transmit a deflate signal. The deflate signal, when received at the electronic pump device 106, causes the electronic pump device 106 to deflate the inflatable member 104 by activating one or more pumps and valves to transfer fluid from the inflatable member 104 to the fluid reservoir 102.

In some examples, the notification manager 128 is configured to inform the user of device statuses 127, including mode changes and fault conditions. For example, the inflatable medical device 100 may operate in a plurality of operating modes such as a storage mode, hibernation mode, MRI mode, end-of-life mode, or normal mode. Some of the operating modes may disable some or all of the features of the inflatable medical device 100. For example, when the inflatable medical device 100 is in the MRI mode, the inflatable member 104 may be prevented from being inflated. In some examples, the modes of the inflatable medical device 100 may be set or adjusted using a setting interface with the settings 139. In some examples, the electronic pump device 106 may programmatically change an operating mode, e.g., switch from a normal mode to a hibernation mode or an end-of-life mode.

In some examples, the notification manager 128 may detect a mode change, and, in response to the detection of the mode change, the notification manager 128 may generate and transmit, via the antenna 188, a mode change signal to the external device 101. The mode change signal, when received at the external device 101, is configured to cause a mode notification 123 to be displayed on the display 144 of the external device 101. For example, the mode change signal causes the client application 140 to generate a mode notification 123, which is displayed on the device's display 144. In some examples, the mode notification 123 is a push notification. The mode notification 123 may include information about the change of operating mode, e.g., the operating mode was changed from a first operating mode to a second operating mode. In some examples, in response to selection of the mode notification or a user control on the mode notification, the external device 101 may cause the client application 140 to be launched, and to display a setting interface to enable the user to switch operating modes.

In some examples, the notification manager 128 may detect a fault condition of the inflatable medical device 100, and, in response to the detection of the fault condition, the notification manager 128 may generate and transmit, via the antenna 188, a fault condition signal to the external device 101. The fault condition signal, when received at the external device 101, is configured to cause a fault notification 125 to be displayed on the display 144 of the external device 101. For example, the fault condition signal causes the client application 140 to generate a fault notification 125, which is displayed on the device's display 144. In some examples, the fault notification 125 is a push notification. The fault notification 125 may include information about the fault condition. In some examples, the fault notification 125 identifies the fault condition, one or more device identifiers about the inflatable medical device 100, and/or patient information about the patient. In some examples, the fault notification 125 includes a fault code. In some examples, in response to selection to the fault notification 125 or selection of the fault code, the external device 101 may cause the client application 140 to launch, and to display a support interface 137 with additional details about the fault condition.

In some examples, depending on the type of fault condition, the notification manager 128 may inform one or more entities of a fault condition. For some fault conditions, the notification manager 128 may transmit a fault notification 125 to the patient and one or more clinicians. For example, the notification manager 128 may transmit a fault notification 125 to the external device 101 and one or more external devices 101a associated with one or more clinicians. In some examples, the notification manager 128 or client application 140 may update a user account 157 with a fault notification 125. In some examples, the medical device platform 152 may transmit information about the fault notification 125 to one or more clinicians, e.g., via email, text, or a notification in the client application 140a. In some examples, a fault notification 125 pertaining to a patient is displayed/viewable in a notification section of the client application 140a.

In some examples, the support interface 137 includes a troubleshooting interface with a search field that enables the user to search troubleshooting information about one or more problems associated with the inflatable medical device 100. In some examples, a user may enter a fault code into a search field of the support interface 137, which may return troubleshooting information about the underlying fault condition. In some examples, in response to selection of the fault notification 125 or selection of the fault code on the fault notification 125, the client application 140 may render the support interface 137 and programmatically enter the fault code into the search field, which automatically returns information about the fault condition. In some examples, the support interface 137 includes customer service contact or physician contact information, a frequently asked questions section, and/or tutorials on how to use the client application 140 (e.g., either in video or written mode). Also, as indicated above, the support interface 137 may include a searchable index for potential codes that are displayed or simply a translation of those codes into actions of which are displayed.

In some examples, the client application 140 includes a chat interface 143 that enables a user to chat with a troubleshooting agent or a language model 172. In some examples, the language model 172 is a predefined large language model or a specially trained language model configured to answer queries about inflatable medical devices 100. For example, a user may enter a user prompt 145 in the chat interface 143. In some examples, the client application 140 may transmit, over the network 150, the user prompt 145 to a prompt manager 162 at the medical device platform 152. The prompt manager 162 may generate a prompt 170, which is provided to the language model 172. In some examples, the prompt 170 includes the user prompt 145. In some examples, the prompt manager 162 may use the user prompt 145 to search and retrieve context data from a context database 168, where the prompt 170 includes the user prompt 145 and the context data. The context database 168 may be a collection of documents about the inflatable medical device 100, such as user manuals, product documents, internal documents, or other documents about inflatable medical devices 100. The retrieved context data may be semantically similar to the user prompt 145 and is used as context for generating a model response 174 by the language model 172. In response to the prompt 170, the language model 172 generates a model response 174 that responds to the user prompt 145 and returns the model response 174 to the prompt manager 162. The prompt manager 162 transmits the model response 174 to the client application 140 for display in the chat interface 143.

In some examples, the client application 140 includes a communication test manager 142 configured to execute a network connection test between the components of the system, e.g., the medical device platform 152, the external device 101, and the electronic pump device 106. In some examples, the client application 140 may display a user control 185 relating to an initiation of the network connection test. In response to selection of the user control 185, the communication test manager 142 may evaluate a first network connection between the external device 101 and the electronic pump device 106 and a second network connection between the external device 101 and the medical device platform 152.

The communication test manager 142 may display the network test results on the user interface 148, which may indicate whether the first and/or second network connections are established or disconnected. If the network test results indicate that a particular network connection has failed, the network test results may include a control or a link, which, when selected, may render a network setting interface to check the network connections. In some examples, the communication test manager 142 may implement logic that can identify one or more configuration settings that caused a network connection to fail, which, when selected, may cause the client application 140 to render a network interface with the configuration setting(s) along with information that identifies the reason of the failure or the correct configuration setting(s). In some examples, the network test results may identify a fault code, which, when selected, causes the display of a support interface 137 to retrieve further information about the fault code and/or initiate a chat interface 143 to chat with a language model 172.

The electronic pump device 106 includes a battery manager 132 configured to notify a user of a battery status 131 and/or a charging status 133. For example, the battery manager 132 may detect a battery status 131 about a charge level of a battery 112 of the electronic pump device 106 or a charging status 133 of the battery 112. In some examples, the battery status 131 is a state of charge of the battery 112. In some examples, the charging status 133 may indicate whether or not the battery 112 is being charged, and, if so, an estimated time of being fully charged. In some examples, the charging status 133 may indicate a level of charging power being delivered to a receiver coil of the electronic pump device 106. For example, a charger device 184 may use wireless charging (e.g., inductive charging, capacitive charging) that transfers electromagnetic energy from a transmitter coil in the charger device 184 to a receiver coil in the electronic pump device 106. The strength of the charging may depend on the alignment of the transmitter coil with the receiver coil. For example, a user may hold the charger device 184 to a position on the user's body (outside of the body) that generally aligns with a position of the electronic pump device 106 that is located inside of the body, but a misalignment may cause the charger power to decrease.

The battery manager 132 may transmit, via the antenna 188, the battery status 131 to the client application 140. The client application 140 displays the battery status 131 on the user interface 148. In some examples, the battery status 131 is displayed as a percentage of state of charge (e.g., 5%, 50%, 100% etc.). In some examples, the client application 140 displays a battery status icon that indicates a level of charge, and the battery status icon is powered by the battery status 131. In some examples, when the battery 112 is charged by a charger device 184, the battery manager 132 may transmit, via the antenna 188, the charging status 133 to the client application 140 and/or the charger device 184.

The charger device 184 may have a display or a charging indicator that indicates the charging status 133. In some examples, the client application 140 may display the charging status 133 on the user interface 148. In some examples, the charging status 133 indicates whether or not the battery 112 is charging. In some examples, the charger device 184 and/or the client application 140 may use lights (e.g., green for charging, red for not charging) or sounds to indicate the charging status 133. For example, the client application 140 and/or the charger device 184 may use the charging status 133 to generate audio sound (e.g., periodic beeping) that indicates that the battery is charging (and, in some examples, no sound when not charging) and a different audio sound (e.g., rapid beeping) when the charging is complete. In some examples, the charging status 133 may indicate a level of charging power delivered to the electronic power device 106, and the user interface 148 and/or the charger device 184 may display the level of charging power or animate (e.g., configure) a graphic that illustrates the level of charging power. In some examples, the user interface 148 and/or the charger device 184 may display an estimated time for fully charged.

In some examples, a software update 154 can be applied to the device's firmware 110 while the electronic pump device 106 is implanted in the user's body. In some examples, when a software update 154 is available on the medical device platform 152, the medical device platform 152 may transmit the software update 154 to the electronic pump device 106, and the electronic pump device 106 may update the firmware 110 with the software update 154. In some examples, when a software update 154 is available on the medical device platform 152, the client application 140 (or the client application 140a) may retrieve, over the network 150, the software update 154. When the client application 140 (or the client application 140a) is connected to the electronic pump device 106, the client application 140 (or the client application 140a) may transmit the software update 154 to the electronic pump device 106 for installation on the electronic pump device 106. For example, the electronic pump device 106 may receive, via the antenna, the software update 154 from the external device 101 (e.g., the client application 140) and update the firmware 110 using the software update 154.

In some examples, the clinician can perform a leak test that measures and displays pressure readings 176 to detect pressure decay. In some examples, a clinician may use the client application 140a to initiate a leak test by a leak detector 130 of the electronic pump device 106. In some examples, the leak test is initiated during the implant procedure, e.g., the components are implanted but the implant procedure is not completed. During the procedure it could serve as a means to verify that connections have been robustly made and/or if there was any damage to the system from the implantation itself, allowing for correction before completing surgery.

In some examples, the leak test is initiated during a follow-up visit with the clinician. In some examples, in response to selection of a UI control 149 on the user interface 148a of the client application 140a, the client application 140a may generate a leak test activation signal 165. The electronic pump device 106 may receive, via the antenna 188, the leak test activation signal 165. In response to the leak test activation signal 165, the electronic pump device 106 may activate a leak detector 130. The leak detector 130 may inflate the inflatable member 104 to one or more target pressure levels by activating one or more pumps and valves to transfer fluid from the fluid reservoir 102 to the inflatable member 104.

In some examples, once reaching a target pressure, the leak detector 130 causes the inflatable member 104 to remain inflated for a period of time (to check for a slow leak). While the inflatable member 104 is being inflated (and then remains at the inflated state) during the leak test, the leak detector 130 may receive pressure readings 176 from the pressure sensor(s) 119 according to a sampling rate. In some examples, the leak detector 130 receives first pressure readings from a pressure sensor 119 connected to the fluid reservoir 102 and/or second pressure readings from a pressure sensor 119 connected to the inflatable member 104. The leak detector 130 may transmit, via the antenna 188, leak test results 175 to the external device 101a (e.g., the client application 140a). The client application 140a may display the leak test results 175 on the display 144a. In some examples, the leak test results 175 includes the pressure readings 176. In some examples, the pressure readings 176 include the first pressure readings. In some examples, the pressure readings 176 include the second pressure readings. In some examples, the pressure readings 176 include the first pressure readings and the second pressure readings. In some examples, a clinician may review the pressure readings 176 to determine whether the inflatable medical device 100 has a leak.

In some examples, the leak detector 130 includes an analyzer that analyzes the pressure readings 176 to determine whether or not the inflatable medical device 100 includes a leak, and, if so, identifies a leak source 178 (e.g., the inflatable member 104, the fluid reservoir 102, one of the tube members 103, 105, or connectors, etc.). In some examples, the leak test results 175 include information that identifies whether or there is a leak, and, if so, information that identifies the leak source 178.

In some examples, the client application 140a receives the pressure readings 176, and the client application 140a includes an analyzer that analyzes the pressure readings 176 to determine whether or not the inflatable medical device 100 includes a leak, and, if so, identifies a leak source 178 (e.g., the inflatable member 104, the fluid reservoir 102, one of the tube members 103, 105, or connectors, etc.). In some examples, the client application 140a receives the pressure readings 176 from the electronic pump device 106 and transmits the pressure readings 176 to the medical device platform 152, which analyzes the pressure readings 176 and returns information with the leak test results 175. In some examples, the pressure readings 176 are transmitted from the electronic pump device 106 to the medical device platform 152, and the medical device platform 152 returns the leak test results 175 to the client application 140a.

In some examples, a clinician may use the client application 140a to view charging trends 171 and/or graphic(s) 173 that depict the charging trends 171 of a user of the inflatable medical device 100. For example, as the electronic pump device 106 is charged using a charger device 184, the electronic pump device 106 may generate and store charging data 120 about the charging of the electronic pump device 106. The electronic pump device 106 may store information about each charging session, which is initiated when a charger device 184 starts to charge the electronic pump device 106. The charging data 120 may include, for each charge session, the charging start time, the charging end time, the charge level at the beginning of the session, the charge level at the end of the session, the charge duration, whether the charge session was initiated after a charging notification, and/or the charge strength, etc. The charging data 120 may be stored in the memory device 109.

In some examples, the electronic pump device 106 may transmit, via the antenna 188, the charging data 120 to the client application 140a for display. In some examples, the clinician can use the client application 140a to retrieve the charging data 120 from the memory device 109, which can be displayed on the display 144a. In some examples, the client application 140a or the client application 140a may retrieve the charging data 120 from the memory device 109 and upload the charging data 120 for storage in the database 155, which is stored under the patient's user account 157. In some examples, when the electronic pump device 106 is connected to the medical device platform 152, the electronic pump device 106 can periodically transmit the charging data 120 to the medical device platform 152, which can be stored in the database 155 in conjunction with the patient's user account 157. In some examples, the client application 140 or the client application 140a may obtain the charging data 120 from the database 155 for display on the user interface 148 or the user interface 148a.

In some examples, the client application 140 or the client application 140 includes a charge trend analyzer configured to analyze the charging data 120 to generate one or more charging trends 171 such as the number of charging times per day/week/month, the average duration, the times of day, etc. The charging trends 171 may include a device charge level at the start of charge, a device charge level at the end of charge session, a rate of charge (e.g., indicates potential alignment of depth of device information as well), and/or time since last charge. The charging trends 171 may include one or more graphics 173 such as graphs that depict the charging trends 171. In some examples, the medical device platform 152 includes a charge trend analyzer configured to analyze the charging data 120 to generate one or more charging trends 171, and the medical device platform 152 may transmit the charging trend(s) 171 for display on the client application 140 or the client application 140a.

In some examples, a clinician or user may use the client application 140a or the client application 140 to view usage trends 141 of the use of the inflatable medical device 100. For example, as a user uses the inflatable medical device 100, the electronic pump device 106 may generate and store usage data 122 about the use of the inflatable medical device 100. The usage data 122 may include a frequency of use, the inflation pressure(s), length of inflation, time of inflation, as well as any user preferred pressure levels in the inflated state and deflated state. In some examples, the usage data 122 may include one or more pressure values that the device experiences, such as peak spike pressures during usage. In some examples, the usage data 122 may include, for each use (e.g., each time the inflatable member 104 was inflated by the patient), the time/date of activating inflation, the target pressure, the length of time to reach target inflated pressure, length of being in the inflated state, the time/date of activating deflation, the deflated state pressure, the length of time to reach target deflated pressure, and/or the values of the settings 139 and/or whether any adjustments to the settings 139 were made.

In some examples, the electronic pump device 106 may transmit, via the antenna 188, the usage data 122 to the client application 140a or the client application 140a for display. In some examples, the clinician or a user can use the client application 140a or the client application 140 to retrieve the usage data 122 from the memory device 109, which can be displayed on the display 144a or the display 144. In some examples, the client application 140a or the client application 140a may retrieve the usage data 122 from the memory device 109 and upload the usage data 122 for storage in the database 155, which is stored under the patient's user account 157. In some examples, when the electronic pump device 106 is connected to the medical device platform 152, the electronic pump device 106 can periodically transmit the usage data 122 to the medical device platform 152, which can be stored in the database 155 in conjunction with the patient's user account 157. In some examples, the client application 140 or the client application 140a may obtain the usage data 122 from the database 155 for display on the user interface 148 or the user interface 148a.

In some examples, the client application 140 or the client application 140 includes a usage trend analyzer configured to analyze the usage data 122 to generate one or more usage trends 141. The usage trends 141 may include information about a frequency of use (e.g., how often they use the device), inflation frequency (e.g., how long they keep the device inflated), protocol adherence (e.g., adherence to prescribed protocols by their physician), changes in utilization of the device, either in frequency or pressures. The usage trends 141 may include one or more graphics (e.g., graphs) that depict the usage trends 141. In some examples, the medical device platform 152 includes a usage trend analyzer configured to analyze the usage data 122 to generate one or more usage trends 141, and the medical device platform 152 may transmit the usage trend(s) 141 for display on the client application 140 or the client application 140a.

In some examples, the electronic pump device 106 may generate and store diagnostic data 116 and performance data 118 in the memory device 109. The performance data 118 may include any information explained with respect to the usage data 122, as well as patient provided feedback (e.g., feedback submitted via the client application 140) regarding erection quality, patient satisfaction, or other factors. In some examples, the performance data 118 may include such as how often it is used, what settings 139 the user selected, adjustments made to settings 139 during usage, or external environments that impact the device such as pressure spikes or changes in elevation. In some examples, the performance data 118 may include changes in inflation time (e.g., over a period of time) or changes in deflation time (e.g., over a period of time) to determine whether performance is degrading. In some examples, the performance data 118 may include the level of maintenance of pressure when inflated (e.g., does the device hold the pressure at the target pressure). Changes in pressures at given inflation time levels may indicate that the device is improving the quality of the erection in that it is stretching tissue leading to some restoration of lost girth or length.

In some examples, the diagnostic data 116 may include error or fault conditions and/or executed calibrations. The diagnostic data 116 may include results of a leak test to check integrity of the system. In some examples, the diagnostic data 116 may include battery performance data or results of a battery check to determine whether the device is degrading, thereby leading to a reduced number of usages per charge. In some examples, the electronic pump device 106 may periodically transmit the diagnostic data 116 and performance data 118 to the medical device platform 152 for storage in the database 155. In some examples, the electronic pump device 106 may periodically transmit the diagnostic data 116 and performance data 118 to the client application 140 or the client application 140a, and the client application 140 or the client application 140a uploads the diagnostic data 116 and performance data 118 to the medical device platform 152 for storage in the database 155.

In some examples, the medical device platform 152 includes an analytics engine 156 configured to receive and analyze the diagnostic data 116 and the performance data 118 to generate analyzed results 166 to be displayed on the user interface 148a and/or execute one or more computer actions 164. In some examples, the analyzed results 166 may include information about if a drop in measured pressure is due to activity (e.g., cyclical pressure waves or relaxation of the inflatable device) and decoupling of these measurements. In some examples, the analyzed results 166 includes information about whether there has a been a change in altitude. The computer actions 164 may include initiating a change to a setting 139, activating one or more pumps to transfer fluid into or out of the inflatable member 104, and/or generating and sending an alert. For example, the medical device platform 152 or the electronic pump device 106 may use pressure measurements to initiate the pump to compensate for the pressure drop due (e.g., due only) to relaxation, thereby maintaining the initial set pressure of the inflatable member 104. If an altitude change has been detected, the medical device platform 152 or the electronic pump device 106 may perform the action to compensate of this change in the patient's selected settings by adjusting the pressure in the inflatable member either by pumping into or out of the inflatable member 104.

In some examples, the analytics engine 156 includes one or more machine-learning models 158. In some examples, the diagnostic data 116 and the performance data 118 are anonymized (e.g., personal information is removed and/or anonymized) and stored in a separate partition in the database 155. In some examples, the database partition may store diagnostic data 116 and performance data 118 across a plurality of inflatable medical devices 100 implanted into patients and across a variety of different models for the inflatable medical devices 100. In some examples, the analytics engine 156 may receive diagnostic data 116 and the performance data 118 of a plurality of inflatable medical devices 100 and generate analyzed results 166. In some examples, the analytics engine 156 may receive diagnostic data 116, performance data 118, charging data 120, usage data, and device data 124, and generate analyzed results 166 based on an analysis of this information.

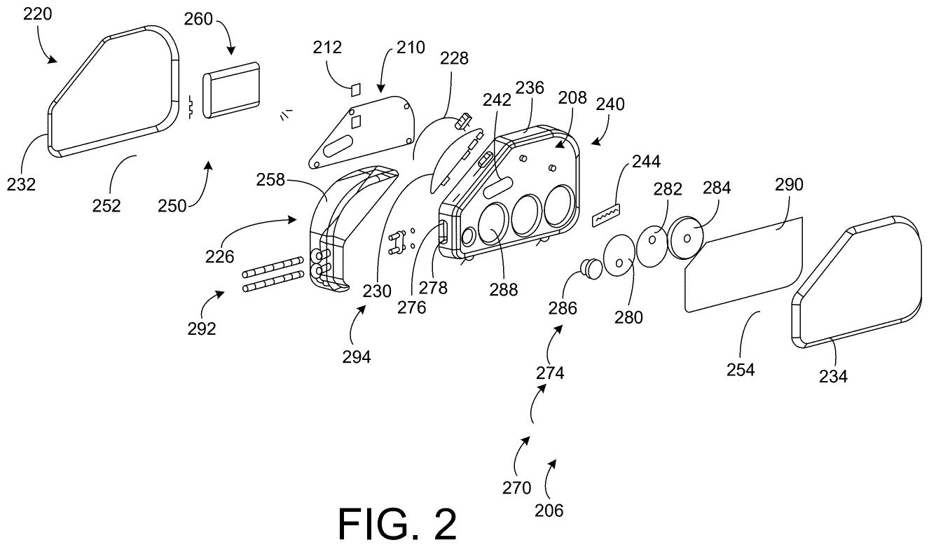

FIG. 2 illustrates an example of an exploded view of an electronic pump device 206 according to an aspect. The electronic pump device 206 includes a housing 220 with a first sidewall 232, a second sidewall 234, a peripheral wall 236, and a frame 240. The first sidewall 232, second sidewall 234, and the peripheral wall 236 are hermetically sealed together to form an internal compartment 250 within the housing 220.

The frame 240 is disposed within the internal compartment 250 to form a first partition 252 and a second partition 254 in such a manner that the first partition 252 is hermetically sealed from the second partition 254. The frame 240 can be integrally formed with the peripheral wall 236, the first sidewall 232, and/or the second sidewall 234. In some examples, the frame 240 is welded to the peripheral wall 236 or welded to the first sidewall 232, and/or the second sidewall 234. The first sidewall 232, the peripheral wall 236, and the frame 240 may form the first partition 252. The second sidewall 234, the peripheral wall 236, and the frame 240 may form the second partition 254, which is opposite the frame 240 from the first partition 252.

The electronic pump device 206 can include a header 226 attached to the housing 220 to form an internal region 258 between an inner surface of the header 226 and an outer surface of the housing 220 that includes power and communication interface structures such as a secondary coil 228 and the antenna 230 external to the hermetically sealed housing 220. The header 226 is configured from a dielectric or insulative material, such as a radome, to allow the transmission of power and communication signals between the antenna 230 and a handset programmer or charger, and between the secondary coil 228 and the charger. For example, the header 226 may include an over molded polymer affixed to the housing 220 and including the secondary coil 228 and the antenna 230 within the internal region 258. The secondary coil 228 and antenna 230 are constructed from a biocompatible material. In some examples, the secondary coil 228 and antenna 230 can be formed as a coil from a stamped titanium core clad with gold or silver. In some examples, the secondary coil 228 and antenna 230 can be formed from a gold wire.

The electronic pump device 206 includes an energy storage system, such as a battery (e.g., a rechargeable power source) (e.g., a rechargeable battery), and electronic components 212 within the first partition 252. The electronic components 212 can be disposed on a circuit substrate 210, such as a plurality of circuit boards, within the first partition 252. The battery 260 can assume various forms appropriate to provide power for generating desired electrical signals and to store power provided from the electronic components 212. For example, the battery 260 can incorporate lithium-ion (Li+) chemistry, e.g., a lithium-ion battery to operate the electronic components 212. In some examples, the electronic components 212 can be implemented by various components including resistors, capacitors, transistors, and integrated circuits disposed on the circuit substrate 210. The secondary coil 228 and antenna 230 are electrically coupled to the electronic components 212 within the first partition 252, such as via a hermetic feedthrough component.

The electronic components 212 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., handset programmers). The communication system can include an integrated circuit to implement an applied communication technology. In some examples, the communication system can be used to transmit communication signals to other devices, such as a charger or the handheld programmer (e.g., external device), and the communication system can be implemented to generate communication signals and provide the communication signals to the antenna 230 for transmission. In some examples, the communication system can be configured to receive and transmit radio frequency signals via the antenna 230. The controller can include a microcontroller to operate the recharge system and to receive and operate in response to communication signals or generate communication signals from the communication system.

The electronic pump device 206 also includes a fluidic circuit 270 within the second partition 254 and opposite the frame 240 from the battery 260 and electronic components 212. In some examples, the frame 240 can include an opening 242 that includes a hermetic interface 244, such as a feedthrough hermetically affixed to the frame 240. The electronic components 212 are operably coupled to the fluidic circuit 270 across the frame 240 via the hermetic interface 244. For example, the controller of the electronic components 212, powered by the battery 260, can cause the operation of the fluidic circuit 270 such as to control and monitor the fluidic circuit 270.

The fluidic circuit 270 includes a fluidic manifold 208 and fluidic components 274 operably coupled to the fluidic manifold 208. In some examples, the fluidic manifold 208 is a structure integrated into the frame 240 such that the fluidic manifold 208 and the frame 240 together form the hermetic barrier between the first partition 252 and the second partition 254 of the internal compartment 250. For instance, the battery 260, the circuit substrate 210, or electronic components 212 can be coupled to a first major surface of the fluidic manifold 208 in the first partition 252, and the fluidic components 274 are operably coupled to a second, and opposite major surface of the fluidic manifold 208 in the second partition 254.

The fluidic circuit 270 provides for the transfer of the fluid between the fluid reservoir (e.g., the fluid reservoir 102 of FIGS. 1A to 1C) and the inflatable member (e.g., the inflatable member 104 of FIGS. 1A to 1C). The fluidic manifold 208, which can be a hermetic manifold, segments and contains the fluid from the internal compartment 250 to reduce the chance of fluid exchange and directs the fluid from a first port 276 to a second port 278 via internal fluid passageways or channels.

The fluidic components 274 include a plurality of fluid pumps, such as pumps 280, 282, a valve 284 mounted into the fluidic manifold 208 in fluidic communication with a manifold passageway to transfer fluid from the first port 276 to the second port 278. The pumps and the valve(s) are in fluid communication with a single fluid passageway between ports 276, 278. The fluidic components 274 also includes one or more pressure sensors 286 operably coupled to the fluidic manifold 208 and in fluidic communication with the passageway to detect a pressure of the fluid within the fluidic manifold 208.

The fluidic components 274 are included in a planar configuration on the fluidic manifold 208 in which the pumps 280, 282, valve 284, and pressure sensor 286 are mounted into the fluidic manifold 208 on a plane for slim profile within the second partition 254. The fluidic manifold 208 can include chambers 288 formed into the second major surface in which the chambers are fluidically coupled to the single passageway within the fluidic manifold 208. The chambers are configured to receive the pumps 280, 282, and valve 284 and one or more pressure sensors 286. In some examples, the fluidic manifold 208 can receive a piezoelectric pump. The fluidic manifold 208 can receive a component cover 290 over the fluidic components 274, which can be hermetically sealed to the second major surface.

In some examples, the electronic pump device 206 may include kink resistant tubing 292 that can extend through the header 226 and attach to the ports 276, 278 via components such as a barb 294 and O-rings. The kink resistant tubing 292 can be attached to the tube members (e.g., tube members 103, 105 of FIGS. 1A to 1C) to fluidically couple the electronic pump device 206 to the fluid reservoir and the inflatable member.

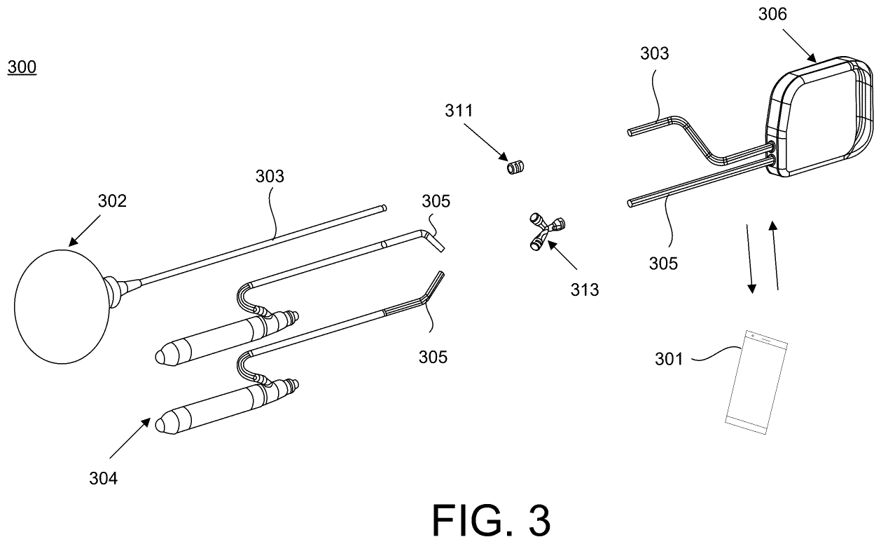

FIG. 3 illustrates a perspective of an inflatable penile prosthesis 300 according to an aspect. The inflatable penile prosthesis 300 may be an example of any of the medical devices discussed herein (e.g., including inflatable medical device 100), and, therefore, may include any of the details discussed with reference to the previous figures.

The inflatable penile prosthesis 300 includes an inflatable member 304, a fluid reservoir 302, and an electronic pump device 306. The inflatable member 304 includes a pair of inflatable cylinders. The electronic pump device 306 may be an example of any of the pump devices discussed with reference to the previous figures and may include any of the details discussed herein. The electronic pump device 306 includes fluidic components such as pumps, valves, and/or sensing devices positioned in fluid passageways. The electronic pump device 306 includes components such as, for example, one or more fluid control devices, one or more pressure sensors, and other such components. The electronic pump device 306 includes an electronic control system configured to provide for the transfer of fluid between a fluid reservoir 302 and an inflatable member 304 via the fluidic components.

The electronic pump device 306 may include one or more integrated circuits. In some examples, the integrated circuits are included in a printed circuit board that is included in a housing of the electronic pump device 306. Fluidic components and the electronic components of the electronic pump device 306 are included in a housing. In some examples, fluidic components and electronic components in the housing define a manifold (e.g., an electronically controlled fluidic manifold) that provides for the electronic control of the flow of fluid between the fluid reservoir 302 and the inflatable member 304. In some examples, the electronic pump device 306 can communicate with an external device 301, via respective communication modules. For example, an application stored in a memory and executed by a processor of the external device 301 may allow the user and/or a physician to operate, view, monitor and alter operation of the inflatable penile prosthesis 300.

The inflatable penile prosthesis 300 includes one or more first tube members 303 that connect a first fluid port of the electronic pump device 306 with the fluid reservoir 302. One or more second tube members 305 connect a second fluid port of the electronic pump device 306 with the inflatable member 304 in the form of the inflatable cylinders. In some examples, the inflatable penile prosthesis 300 includes a connector 311 that is used to connect two tube members 303 together, and a connector 313 that is used to connect two tube members 305 together.

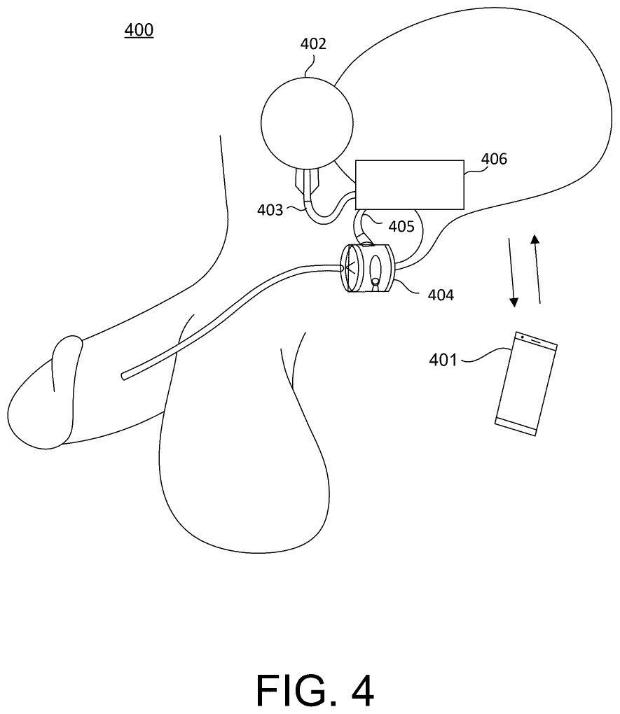

FIG. 4 illustrates a urinary control device 400 having an electronic pump device 406 according to an aspect. The urinary control device 400 may be an example of the inflatable medical device 100. In some examples, the urinary control device 400 is an artificial urinary sphincter device. The electronic pump device 406 may include any of the features of the pump devices discussed herein. The urinary control device 400 includes an electronic pump device 406, a fluid reservoir 402, and a cuff 404 (e.g., an inflatable cuff).

The fluid reservoir 402 may be a pressure-regulating inflation balloon or element. The fluid reservoir 402 is in operative fluid communication with the cuff 404 via one or more tube members 403, 405. The fluid reservoir 402 is constructed of polymer material that is capable of elastic deformation to reduce fluid volume within the fluid reservoir 402 and push fluid out of the fluid reservoir 402 and into the cuff 404. However, the material of the fluid reservoir 402 can be biased or include a shape memory construct adapted to generally maintain the fluid reservoir 402 in its expanded state with a relatively constant fluid volume and pressure. In some examples, this constant level of pressure exerted from the fluid reservoir 402 to the cuff 404 will keep the cuff 404 at a desired inflated state when open fluid communication is provided between the fluid reservoir 402 and the cuff 404. In some examples, the fluid reservoir 402 is implanted into the abdominal space.

A user may use an external device 401 to control the urinary control device 400. In some examples, the user may use the external device 401 to inflate or deflate the cuff 404. For example, in response to the user activating an inflation cycle using the external device 401, the external device 401 may transmit a wireless signal to the electronic pump device 406 to initiate the inflation cycle to transfer fluid from the fluid reservoir 402 to the cuff 404 (e.g., by opening an active valve where the pressure in the fluid reservoir 402 causes the fluid to move through the active valve to the cuff 404). In some examples, in response to the user activating a deflation cycle using the external device 401, the external device 401 may transmit a wireless signal to the pump device 406 to initiate the deflation cycle to transfer fluid from the cuff 404 to the fluid reservoir 402.

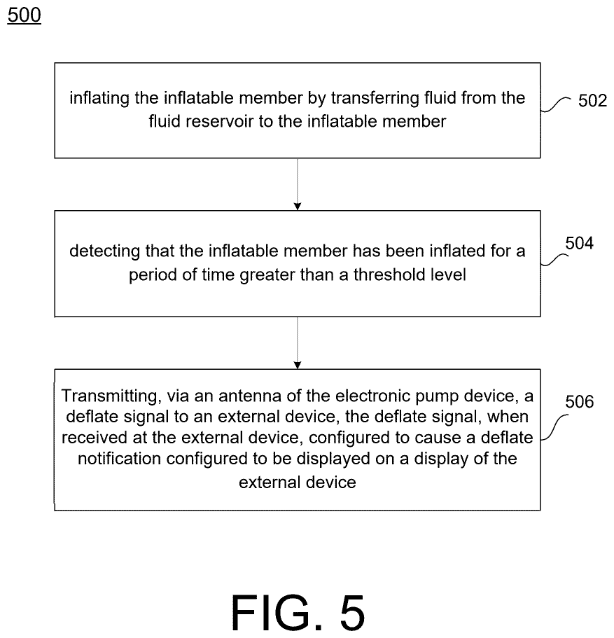

FIG. 5 illustrates a flowchart 500 depicting example operations of implementing one or more troubleshooting features of an inflatable medical device. Although the flowchart 500 of FIG. 5 illustrates the operations in sequential order, it will be appreciated that this is merely an example, and that additional or alternative operations may be included. Further, operations of FIG. 5 and related operations may be executed in a different order than that shown, or in a parallel or overlapping fashion.

Operation 502 includes inflating the inflatable member by transferring fluid from the fluid reservoir to the inflatable member. Operation 504 includes detecting that the inflatable member has been inflated for a period of time greater than a threshold level. Operation 506 includes transmitting, via an antenna of the electronic pump device, a deflate signal to an external device, the deflate signal, when received at the external device, configured to cause a deflate notification configured to be displayed on a display of the external device.

Clause 1. An inflatable medical device comprising: an inflatable member; a fluid reservoir; and an electronic pump device configured to be fluidly connected to the inflatable member and the fluid reservoir, the electronic pump device configured to execute operations, the operations comprising: inflating the inflatable member by transferring fluid from the fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

Clause 2. The inflatable medical device of clause 1, wherein, in response to the inflatable member being detected for the period of time greater than the threshold level, initiating deflation of the inflatable member.

Clause 3. The inflatable medical device of clause 1 or 2, wherein the external device is a first external device, wherein the operations comprise: receiving a leak test activation signal from a second external device associated with a clinician; in response to the leak test activation signal, inflating the inflatable member by transferring fluid from the fluid reservoir to the inflatable member; generating pressure readings of at least one of the fluid reservoir or the inflatable member; and transmitting, via the antenna of the electronic pump device, leak test results to the second external device, the leak test results including the pressure readings.

Clause 4. The inflatable medical device of clause 3, wherein the operations further comprise: receiving the leak test activation signal during an implantation procedure of the inflatable medical device.

Clause 5. The inflatable medical device of any one of clauses 1 to 4, wherein the operations further comprise: detecting a fault condition of the inflatable medical device; and transmitting, via the antenna of the electronic pump device, a device status signal to the external device, the device status signal, when received at the external device, configured to display a fault condition notification about the fault condition on the display of the external device.

Clause 6. The inflatable medical device of any one of clauses 1 to 5, wherein the operations further comprise: detecting at least one of a battery status about a charge level of a battery of the electronic pump device or a charging status of the battery; and transmitting, via the antenna of the electronic pump device, at least one of the battery status or the charging status to the external device for display on the external device.

Clause 7. The inflatable medical device of any one of clauses 1 to 6, wherein the external device is a first external device, wherein the operations further comprise: receiving, via the antenna of the electronic pump device, a software update from the first external device or a second external device associated with a clinician; and updating a firmware of the electronic pump device using the software update.

Clause 8. The inflatable medical device of any one of clauses 1 to 7, wherein the external device is a first external device, wherein the operations further comprise: retrieving, from a memory device of the electronic pump device, at least one of performance data or diagnostic data about the inflatable medical device; and transmitting, via the antenna of the electronic pump device, at least one of the performance data or the diagnostic data to the first external device or a second external device associated with a clinician.

Clause 9. The inflatable medical device of any one of clauses 1 to 8, wherein the external device is a first external device, the inflatable medical device further comprising: a client application executable by the first external device or a second external device associated with a clinician, the client application configured to communicate with the electronic pump device, the client application configured to display one or more device controls for controlling the inflatable medical device.

Clause 10. The inflatable medical device of clause 9, wherein the client application is configured to: receive usage data from the electronic pump device; initiate generation of one or more usage trends using the usage data; and display the one or more usage trends on the display of the first external device or the second external device.

Clause 11. The inflatable medical device of clause 9, wherein the client application is configured to: receive, via a chat interface of the client application, a user prompt for a language model; and display a model response generated by the language model that responds to the user prompt.

Clause 12. The inflatable medical device of clause 9, wherein the client application is configured to receive, via one or more input fields of a user interface of the client application, at least one of patient data about a user of the inflatable medical device or device data about the inflatable medical device; and transmit, over a network, at least one of the patient data or the device data to a medical device platform executable by one or more server computers, the medical device platform configured to store at least one of the patient data or the device data in a database at the medical device platform.

Clause 13. The inflatable medical device of clause 9, wherein the client application is configured to: receive a selection of a network connection test; execute the network connection test to evaluate a network connection between the client application and the electronic pump device and a network connection between the client application and a medical device platform executable by one or more server computers; and display network connection results of the network connection test on a user interface of the client application.

Clause 14. The inflatable medical device of clause 9, wherein the client application is configured to: display a support interface configured to provide troubleshooting information about troubleshooting one or more problems associated with the inflatable medical device.

Clause 15. The inflatable medical device of clause 9, wherein the client application is configured to: receive collected data from the electronic pump device, the collected data including at least one of diagnostic data, performance data, charging data, or usage data; and transmit, over a network, the collected data to a medical device platform executable by one or more server computers, the collected data being stored in a database at the medical device platform.

Clause 16. An inflatable medical device comprising: an inflatable member; a fluid reservoir; and an electronic pump device configured to be fluidly connected to the inflatable member and the fluid reservoir, the electronic pump device configured to execute operations, the operations comprising: inflating the inflatable member by transferring fluid from the fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

Clause 17. The inflatable medical device of clause 16, wherein, in response to the inflatable member being detected for the period of time greater than the threshold level, initiating deflation of the inflatable member.

Clause 18. The inflatable medical device of clause 16, wherein the external device is a first external device, wherein the operations comprise: receiving a leak test activation signal from a second external device associated with a clinician; in response to the leak test activation signal, inflating the inflatable member by transferring fluid from the fluid reservoir to the inflatable member; generating pressure readings of at least one of the fluid reservoir or the inflatable member; and transmitting, via the antenna of the electronic pump device, leak test results to the second external device, the leak test results including the pressure readings.

Clause 19. The inflatable medical device of clause 18, wherein the operations further comprise: receiving the leak test activation signal during an implantation procedure of the inflatable medical device.

Clause 20. The inflatable medical device of clause 16, wherein the operations further comprise: detecting a fault condition of the inflatable medical device; and transmitting, via the antenna of the electronic pump device, a device status signal to the external device, the device status signal, when received at the external device, configured to display a fault condition notification about the fault condition on the display of the external device.

Clause 21. The inflatable medical device of clause 16, wherein the operations further comprise: detecting at least one of a battery status about a charge level of a battery of the electronic pump device or a charging status of the battery; and transmitting, via the antenna of the electronic pump device, at least one of the battery status or the charging status to the external device for display on the external device.

Clause 22. The inflatable medical device of clause 16, wherein the external device is a first external device, wherein the operations further comprise: receiving, via the antenna of the electronic pump device, a software update from the first external device or a second external device associated with a clinician; and updating a firmware of the electronic pump device using the software update.

Clause 23. The inflatable medical device of clause 16, wherein the external device is a first external device, wherein the operations further comprise: retrieving, from a memory device of the electronic pump device, at least one of performance data or diagnostic data about the inflatable medical device; and transmitting, via the antenna of the electronic pump device, at least one of the performance data or the diagnostic data to the first external device or a second external device associated with a clinician.

Clause 24. The inflatable medical device of clause 16, wherein the external device is a first external device, the inflatable medical device further comprising: a client application executable by the first external device or a second external device associated with a clinician, the client application configured to communicate with the electronic pump device, the client application configured to display one or more device controls for controlling the inflatable medical device.

Clause 25. The inflatable medical device of clause 24, wherein the client application is configured to: receive usage data from the electronic pump device; initiate generation of one or more usage trends using the usage data; and display the one or more usage trends on the display of the first external device or the second external device.

Clause 26. The inflatable medical device of clause 24, wherein the client application is configured to: receive, via a chat interface of the client application, a user prompt for a language model; and display a model response generated by the language model that responds to the user prompt.

Clause 27. The inflatable medical device of clause 24, wherein the client application is configured to receive, via one or more input fields of a user interface of the client application, at least one of patient data about a user of the inflatable medical device or device data about the inflatable medical device; and transmit, over a network, at least one of the patient data or the device data to a medical device platform executable by one or more server computers, the medical device platform configured to store at least one of the patient data or the device data in a database at the medical device platform.

Clause 28. The inflatable medical device of clause 24, wherein the client application is configured to: receive a selection of a network connection test; execute the network connection test to evaluate a network connection between the client application and the electronic pump device and a network connection between the client application and a medical device platform executable by one or more server computers; and display network connection results of the network connection test on a user interface of the client application.

Clause 29. The inflatable medical device of clause 24, wherein the client application is configured to: display a support interface configured to provide troubleshooting information about troubleshooting one or more problems associated with the inflatable medical device.

Clause 30. The inflatable medical device of clause 24, wherein the client application is configured to: receive collected data from the electronic pump device, the collected data including at least one of diagnostic data, performance data, charging data, or usage data; and transmit, over a network, the collected data to a medical device platform executable by one or more server computers, the collected data being stored in a database at the medical device platform.

Clause 31. A method comprising: inflating, by an electronic pump device, an inflatable member by transferring fluid from a fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

Clause 32. The method of clause 31, wherein the external device is a first external device, the method comprising: receiving, during an implantation procedure, a leak test activation signal from a second external device associated with a clinician; in response to the leak test activation signal, inflating the inflatable member by transferring fluid from the fluid reservoir to the inflatable member; generating pressure readings of at least one of the fluid reservoir or the inflatable member; and transmitting, via the antenna of the electronic pump device, leak test results to the second external device, the leak test results including the pressure readings.

Clause 33. The method of clause 31, further comprising: detecting a mode change; and transmitting, via the antenna of the electronic pump device, a device status signal to the external device, the device status signal, when received at the external device, configured to display a mode change notification about the mode change on the display of the external device.

Clause 34. A non-transitory computer-readable medium storing executable instructions that cause at least one processor to execute operations, the operations comprising: inflating, by an electronic pump device, an inflatable member by transferring fluid from a fluid reservoir to the inflatable member; detecting that the inflatable member has been inflated for a period of time greater than a threshold level; and transmitting, via an antenna of the electronic pump device, a deflate signal to an external device, the deflate signal, when received at the external device, configured to cause a deflate notification configured to be displayed on a display of the external device.

Clause 35. The non-transitory computer-readable medium of clause 34, wherein the external device is a first external device, wherein the operations further comprise: receiving, via the antenna of the electronic pump device, a software update from the first external device or a second external device associated with a clinician; and updating a firmware of the electronic pump device using the software update.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An inflatable medical device comprising:
an inflatable member;
a fluid reservoir; and
an electronic pump device configured to be fluidly connected to the inflatable member and the fluid reservoir, the electronic pump device configured to execute operations, the operations comprising:
inflating the inflatable member by transferring fluid from the fluid reservoir to the inflatable member;
detecting that the inflatable member has been inflated for a period of time greater than a threshold level; and
transmitting, via an antenna of the electronic pump device, a deflate signal to an external device, the deflate signal, when received at the external device, configured to cause a deflate notification configured to be displayed on a display of the external device.

2. The inflatable medical device of claim 1, wherein, in response to the inflatable member being detected for the period of time greater than the threshold level, initiating deflation of the inflatable member.

3. The inflatable medical device of claim 1, wherein the external device is a first external device, wherein the operations comprise:
receiving a leak test activation signal from a second external device associated with a clinician;
in response to the leak test activation signal, inflating the inflatable member by transferring fluid from the fluid reservoir to the inflatable member;
generating pressure readings of at least one of the fluid reservoir or the inflatable member; and
transmitting, via the antenna of the electronic pump device, leak test results to the second external device, the leak test results including the pressure readings.

4. The inflatable medical device of claim 3, wherein the operations further comprise:
receiving the leak test activation signal during an implantation procedure of the inflatable medical device.

5. The inflatable medical device of claim 1, wherein the operations further comprise:
detecting a fault condition of the inflatable medical device; and
transmitting, via the antenna of the electronic pump device, a device status signal to the external device, the device status signal, when received at the external device, configured to display a fault condition notification about the fault condition on the display of the external device.

6. The inflatable medical device of claim 1, wherein the operations further comprise:
detecting at least one of a battery status about a charge level of a battery of the electronic pump device or a charging status of the battery; and
transmitting, via the antenna of the electronic pump device, at least one of the battery status or the charging status to the external device for display on the external device.

7. The inflatable medical device of claim 1, wherein the external device is a first external device, wherein the operations further comprise:
receiving, via the antenna of the electronic pump device, a software update from the first external device or a second external device associated with a clinician; and
updating a firmware of the electronic pump device using the software update.

8. The inflatable medical device of claim 1, wherein the external device is a first external device, wherein the operations further comprise:
retrieving, from a memory device of the electronic pump device, at least one of performance data or diagnostic data about the inflatable medical device; and
transmitting, via the antenna of the electronic pump device, at least one of the performance data or the diagnostic data to the first external device or a second external device associated with a clinician.

9. The inflatable medical device of claim 1, wherein the external device is a first external device, the inflatable medical device further comprising:
a client application executable by the first external device or a second external device associated with a clinician, the client application configured to communicate with the electronic pump device, the client application configured to display one or more device controls for controlling the inflatable medical device.

10. The inflatable medical device of claim 9, wherein the client application is configured to:
receive usage data from the electronic pump device;
initiate generation of one or more usage trends using the usage data; and
display the one or more usage trends on the display of the first external device or the second external device.

11. The inflatable medical device of claim 9, wherein the client application is configured to:
receive, via a chat interface of the client application, a user prompt for a language model; and
display a model response generated by the language model that responds to the user prompt.

12. The inflatable medical device of claim 9, wherein the client application is configured to
receive, via one or more input fields of a user interface of the client application, at least one of patient data about a user of the inflatable medical device or device data about the inflatable medical device; and
transmit, over a network, at least one of the patient data or the device data to a medical device platform executable by one or more server computers, the medical device platform configured to store at least one of the patient data or the device data in a database at the medical device platform.

13. The inflatable medical device of claim 9, wherein the client application is configured to:
receive a selection of a network connection test;
execute the network connection test to evaluate a network connection between the client application and the electronic pump device and a network connection between the client application and a medical device platform executable by one or more server computers; and
display network connection results of the network connection test on a user interface of the client application.

14. The inflatable medical device of claim 9, wherein the client application is configured to:
display a support interface configured to provide troubleshooting information about troubleshooting one or more problems associated with the inflatable medical device.

15. The inflatable medical device of claim 9, wherein the client application is configured to:
receive collected data from the electronic pump device, the collected data including at least one of diagnostic data, performance data, charging data, or usage data; and
transmit, over a network, the collected data to a medical device platform executable by one or more server computers, the collected data being stored in a database at the medical device platform.

16. A method comprising:
inflating, by an electronic pump device, an inflatable member by transferring fluid from a fluid reservoir to the inflatable member;
detecting that the inflatable member has been inflated for a period of time greater than a threshold level; and
transmitting, via an antenna of the electronic pump device, a deflate signal to an external device, the deflate signal, when received at the external device, configured to cause a deflate notification configured to be displayed on a display of the external device.

17. The method of claim 16, wherein the external device is a first external device, the method comprising:
receiving, during an implantation procedure, a leak test activation signal from a second external device associated with a clinician;
in response to the leak test activation signal, inflating the inflatable member by transferring fluid from the fluid reservoir to the inflatable member;
generating pressure readings of at least one of the fluid reservoir or the inflatable member; and
transmitting, via the antenna of the electronic pump device, leak test results to the second external device, the leak test results including the pressure readings.

18. The method of claim 16, further comprising:
detecting a mode change; and
transmitting, via the antenna of the electronic pump device, a device status signal to the external device, the device status signal, when received at the external device, configured to display a mode change notification about the mode change on the display of the external device.

19. A non-transitory computer-readable medium storing executable instructions that cause at least one processor to execute operations, the operations comprising:
inflating, by an electronic pump device, an inflatable member by transferring fluid from a fluid reservoir to the inflatable member;
detecting that the inflatable member has been inflated for a period of time greater than a threshold level; and
transmitting, via an antenna of the electronic pump device, a deflate signal to an external device, the deflate signal, when received at the external device, configured to cause a deflate notification configured to be displayed on a display of the external device.

20. The non-transitory computer-readable medium of claim 19, wherein the external device is a first external device, wherein the operations further comprise:
receiving, via the antenna of the electronic pump device, a software update from the first external device or a second external device associated with a clinician; and
updating a firmware of the electronic pump device using the software update.
